(19) United States
(12) Patent Application Publication
(10) Pub. No.: US 2005/0055015 A1
(43) Pub. Date: Mar. 10, 2005

(54) Laser delivery device incorporationg a plurality of laser source optical fibers

(76) Inventor: David M. Buzawa, San Jose, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/688,069

(22) Filed: Oct. 17, 2003

Related U.S. Application Data
(60) Provisional application No. 60/419,467, filed on Oct. 17, 2002.

(51) Int. Cl. A61B 18/20

(52) U.S. Cl. 606/4; 606/6

(57) ABSTRACT

A laser system has at least a first laser source and a second laser source. At least a first fiber is coupled to the first laser source. At least a second fiber is coupled to the second laser source. A fiber switching device is coupled to the first and second fibers. The fiber switching device is configured to provide laser delivery from each of the first and second fibers without additional optical alignment.

### CROSS-REFERENCE TO RELATED APPLICATIONS

This application claims the benefit of priority of U.S. Provisional Application Ser. No. 60/419,467 filed Oct. 17, 2002, which is fully incorporated herein by reference for all purposes.

### BACKGROUND OF THE INVENTION

1. Field of the Invention

This invention relates generally to a laser delivery device with a multitude of source optical fibers, and more particularly to a laser delivery device where the multiple fibers are connected to, (i) different laser sources in order to facilitate the delivery of laser energy from more than one laser source in succession, or to (ii) a single laser source, in order to facilitate the delivery of laser energy with a wide range of treatment parameters.

2. Description of the Related Art

Lasers have found many useful clinical applications in medicine, and particularly within the specialty of ophthalmology. Today, lasers are used to treat a wide variety of ocular disorders, including pathologies related to diabetes, glaucoma, macular degeneration and intraocular tumors. Such pathology is typically evaluated by an ophthalmologist who often uses a slit lamp biomicroscope to illuminate and observe intraocular anatomy. Such slit lamp biomicroscopes are available commercially from a number of manufacturers, including Haag-Streit and Zeiss.

When medically indicated, the ophthalmologist may also treat a pathology, frequently using for such purposes a slit lamp biomicroscope capable of delivering appropriate therapeutic laser energy. Such a slit lamp laser delivery device (referred to herein as “slit lamp adapter” or “SLA”) is typically comprised of a plurality of laser delivery optical elements aligned to each other and mounted temporarily or permanently onto the slit lamp microscope. Laser energy is transported to this optical train from a compatible laser source via a flexible optical fiber cable, which has been found to be durable, efficient and convenient for this purpose. The optics within the SLA typically collect light emitted from the output face of the optical fiber and project a real image of the fiber face at some convenient distance and predetermined magnification M_{SLA}. Since M_{SLA} is not dependent on fiber core diameter, fibers with larger core diameters will produce proportionally larger images through a given SLA. Depending on the specific SLA design, such a fiber image may be in focus (“parfocal”) or intentionally out of focus at the ophthalmologist's viewing plane.

Typically included within the SLA laser delivery is a means of adjusting the size of the treatment laser spot, permitting the ophthalmologist to conveniently match the observed pathology with appropriately sized laser spots. Such spot size adjustment mechanisms may be continuously variable or discrete in design, offering a number of specific, user-selectable spot sizes. The diameter of such treatment laser spot sizes cover a spot size ratio of from 6 to 1 to perhaps 15 or 20 to 1. A spot size range of ˜0.060-0.500 mm, or an 8:1 range, is a popular specification. This range has overlapped well with most conventional ocular laser therapies to date, including panretinal photocoagulation for diabetic retinopathy, laser trabeculoplasty and laser treatment of diabetic macular edema.

Recently, the advent and rise in popularity of transpupillary thermotherapy (“TTT”) and other large-spot-size low-irradiance ocular laser treatments has created a need for SLA devices with even greater flexibility and spot size range. While conventional laser therapies are frequently performed with visible laser sources and SLA spot size selections of 100-300 micrometers, TTT and other low irradiance therapies are performed with infrared laser sources and multimillimeter spot sizes of up to 5 mm in diameter. No conventional SLA is able to offer, for example, an 80 to 1 (0.060 to 5.00 mm) range of spot size selections in order to simultaneously satisfy the very diverse requirements of these treatments, necessitating the use of multiple separate SLA devices to satisfy all desired clinical applications.

Additionally, a growing number of procedures once performed almost exclusively with conventional (i.e. visible green) laser photocoagulators are instead performed, at least on selected patients, with alternate wavelength lasers, including yellow, orange, red and infrared laser sources. These alternate wavelength lasers often have unique characteristics, such as wavelength, pulse shape, size or efficiency that can be used to advantage by the clinician. Some lasers such as tunable dye lasers or multi-line ion lasers are intrinsically multi-wavelength. In these cases, if a means of controlling the laser wavelength is made available to the clinician, a single SLA connected to such a laser can deliver a multitude of laser wavelengths from that single laser. Other lasers, however, including solid-state 532 nm lasers and laser diodes, are essentially monochromatic, and an SLA connected to a source of this type is limited to delivery of that single treatment laser wavelength. No SLA to date has had the ability to connect to a multitude (more than one) of laser sources to facilitate the delivery of laser light from a variety of discrete sources.

The use of a plurality of fiber optics with differing core dimensions to change retinal spot sizes delivered from a single (argon ion) laser source via a hand-held ophthalmoscope, SLA or indirect ophthalmoscope, has been described by Kapany N. S., Green Laser Photocoagulator Using Fiber Optics, Arch Ophthal 88:80-84, 1972. Switching the input and output ends of the fibers permitted adjustment of delivered spot size, albeit over a somewhat limited range, 4:1 as described in the published article.

There is a need for an improved laser system with a multitude of source optical fibers. There is a further need for a laser system with multiple fibers connected to different laser sources in order to facilitate the delivery of laser energy from more than one laser source in succession. There is yet another need for a laser system with multiple fibers connected to a single laser source, in order to facilitate the delivery of laser energy with a wide range of treatment parameters.

### SUMMARY OF THE INVENTION

Accordingly, an object of the present invention is to provide an improved laser system with a multitude of source optical fibers.

Another object of the present invention is to provide a laser system with multiple fibers connected to different laser sources in order to facilitate the delivery of laser energy from more than one laser source in succession.

Yet another object of the present invention is to provide a laser system with multiple fibers connected to a single laser source, in order to facilitate the delivery of laser energy with a wide range of treatment parameters.

These and other objects of the present invention are achieved in a laser system with at least a first laser source and a second laser source. At least a first fiber is coupled to the first laser source. At least a second fiber is coupled to the second laser source. A fiber switching device is coupled to the first and second fibers. The fiber switching device is configured to provide laser delivery from each of the first and second fibers without additional optical alignment.

In another embodiment of the present invention, a laser system has at least first laser and second laser sources. At least a first fiber is coupled to the first laser source, and at least a second fiber coupled to the second laser source. A fiber switching device is coupled to the first and second fibers. The fiber switching device is configured to provide repositioning of and laser delivery from each of the first and second fibers without additional optical alignment.

In another embodiment of the present invention, a laser delivery device includes a laser source. At least a first fiber is capable of being coupled to the laser source, and at least a second fiber is coupled to the laser source. A fiber switching mechanism is configured to provide laser delivery from each of the first and second fibers without the need for additional optical alignment.

In another embodiment of the present invention, a laser delivery device has a laser source. At least a first and a second fiber are capable of being coupled to the laser source. A fiber switching mechanism is configured to provide repositioning of and laser delivery from each of the first and second fibers without the need for additional optical alignment. A spot size adjustment device is coupled to at least one of the first and second fibers.

### BRIEF DESCRIPTION OF THE FIGURES

FIG. 1 shows a laser system having a single laser source coupled by multiple fibers to a spot size adjustment device.

FIG. 2(a) depicts a laser system having multiple laser sources.

FIG. 2(b) illustrates one embodiment of a fiber sensing device that can be used with the laser systems of FIGS. 1 and 2.(a).

FIG. 3 depicts a further embodiment of the laser system according to the present invention.

FIG. 4 depicts multiple laser sources coupled by fibers of different diameters to a spot size adjustment device.

FIG. 5 depicts a single laser source coupled by multiple fibers of different diameters.

FIG. 6 illustrates one embodiment of a fiber switch coupled to a spot adjustment device in order to provide both course and fine spot size adjustment.

### DESCRIPTION OF THE PREFERRED EMBODIMENTS

In various embodiments, the present invention is a laser delivery device with a multitude of fibers coupled to one or more sources. In one embodiment, the multiple fibers are connected to different laser sources. In another embodiment, the multiple fibers are coupled to a single laser source, either sequentially or simultaneously.

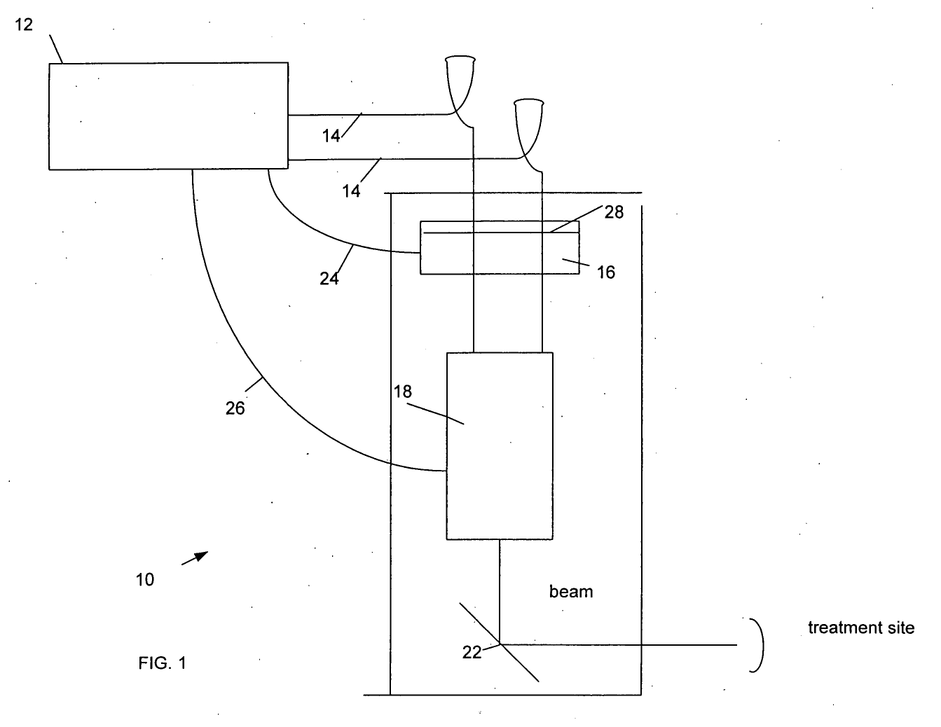

Referring to FIG. 1, one embodiment of a laser system 10 of the present invention includes a single laser source 12 coupled by two optical fibers 14 to a fiber switch 16, which is in turn coupled to spot adjustment device 18. Fiber switch 16, spot adjustment device 18, or both can be a laser delivery device, generally denoted as 20 A fold mirror 22 can be included to direct the beam from laser source 12 to a treatment site. Two or more fibers 14 are coupled to the laser source 12 and to laser delivery device 20. The fibers 14 can have different cross-sectional dimensions in order to permit selection of spot sizes differing by factors of 100 to 1 and more. The fiber 14 that is selected can serve as the coarse range selection mechanism and spot size adjustment device 18 acts as the final spot size determinant. Spot size adjustment device 18 provides automatic fiber sensing at the output and input ends of fibers 14.

A fiber sensor feedback loop 24 can be coupled to laser source 12 and fiber switch 16, and a spot size feedback loop 26 can be coupled to laser source 12 and spot adjustment device 18.

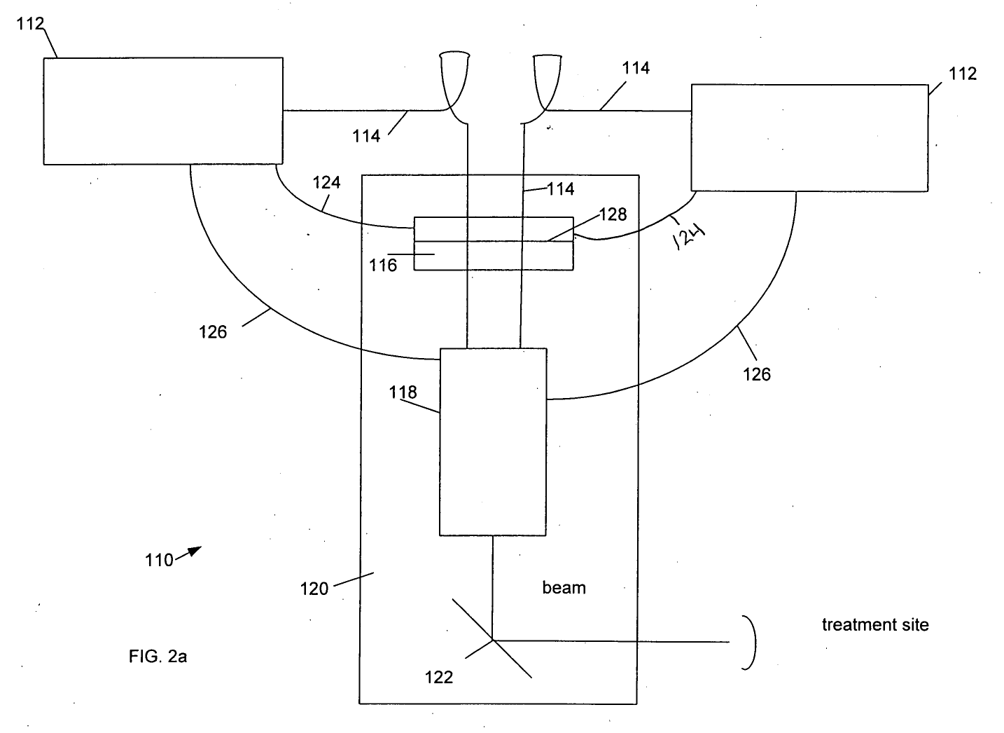

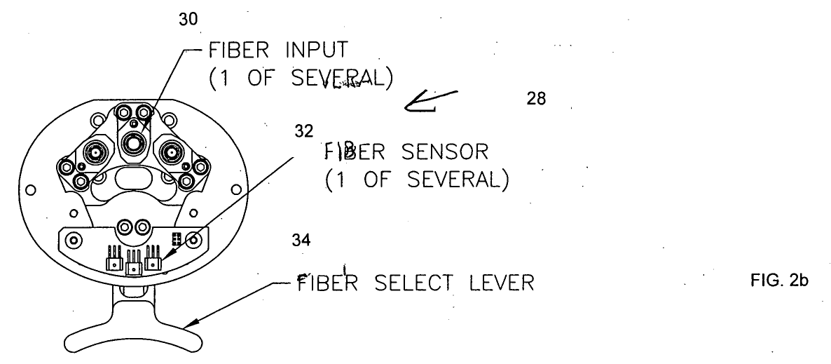

In another embodiment, illustrated in FIG. 2(a), laser system 110 includes at least first and second laser sources 112, each coupled to one or more fibers 114. Each fiber 114 is in turn coupled to a fiber switch 116 and a spot size adjustment device 118. Again, fiber switch 116, spot adjustment device 118, or both can be a laser delivery device, generally denoted as 120 A fold mirror 122 can be included to direct the beam from laser sources 112 to a treatment site. Laser system 110 also includes a fiber sensor feedback loop 124 a spot size feedback loop 126.

Fiber switches 16 and 116 permit selection of a certain fiber 14 or 114 without the need for disconnecting the other fiber(s) 14 and 114. Fiber switches 18 and 118 provide coupling of the fibers 14 and 114 without the need for additional optical alignment elements. Fiber switches 16 and 116 move fibers 14 and 114 over an input lens. In one embodiment, a carousal of multiple fibers 14, 114 is provided. The carousal of fibers moves over the input lens. It will be appreciated that fiber switches 16 and 116 can implement different methods of switching the fibers 14 and 114, including but not limited to moving the input lens, instead of the fibers 14 and 114. Additionally, fiber switches 16 and 116 provide for rapid change for the selection of the laser source 12 and 112. This permits change in selection of spot size and other related treatment parameters that can readily be linked to the selection of the laser source 12 and 112, including exposure duration and repetitive pulse frequency.

Fiber switches 16 and 116 can include a fiber sensing device 28 and 128 respectively, illustrated in FIG. 2(b). In one embodiment, fiber sensing device 28 and 128 each include one or more fiber inputs 30, one or more fiber sensors 32 and a fiber select lever 34.

The present invention, including but not limited to laser systems 10 and 110, can be a device suitable for medical applications compatible with fiber optics. Suitable medical applications include but not limited to, (i) laser photocoagulation as performed in ophthalmology, dermatology, otology, urology, gynecology and other medical specialties, (ii) laser ablation as performed in urology, orthopedic surgery, ENT surgery, neurosurgery, general surgery and other medical specialties, (iii) photodynamic therapy as performed in ophthalmology and oncology, and (iv) hyperthermia, transpupillary thermotherapy, biostimulation and other such applications generally characterized by large spot size, low irradiance laser treatment parameters, and the like.

In various embodiments, the laser sources useful with the present invention, including but not limited to laser sources 12, and 112, can be selected from a variety of different lasers. Such suitable lasers may include but are not limited to, diode, ion, dye, Ti:sapphire, Alexandrite, solid state and the like. A variety of different host materials can be utilized with the solid state lasers including but not limited to, YAG, YVO4 or YSGG doped with rare earth elements such as Nd, Yb, Er, Ho, Tm, and the like

Examples of suitable laser delivery devices 20 and 120 include but are not limited to devices that are used in the field of ophthalmology include such as a, laser slit lamp adapter, as well as any ophthalmic device that may potentially be adaptable to and convenient for laser delivery, such as an indirect ophthalmoscope, laser operating microscope, direct ophthalmoscope, intraocular probe, scanning laser ophthalmoscope, fundus camera and the like. Examples of laser delivers devices 20 and 120 that can be used in non-ophthalmic medical specialties include but are not limited to, laparascopes, endoscopes, microscopes, various handheld laser delivery devices and the like.

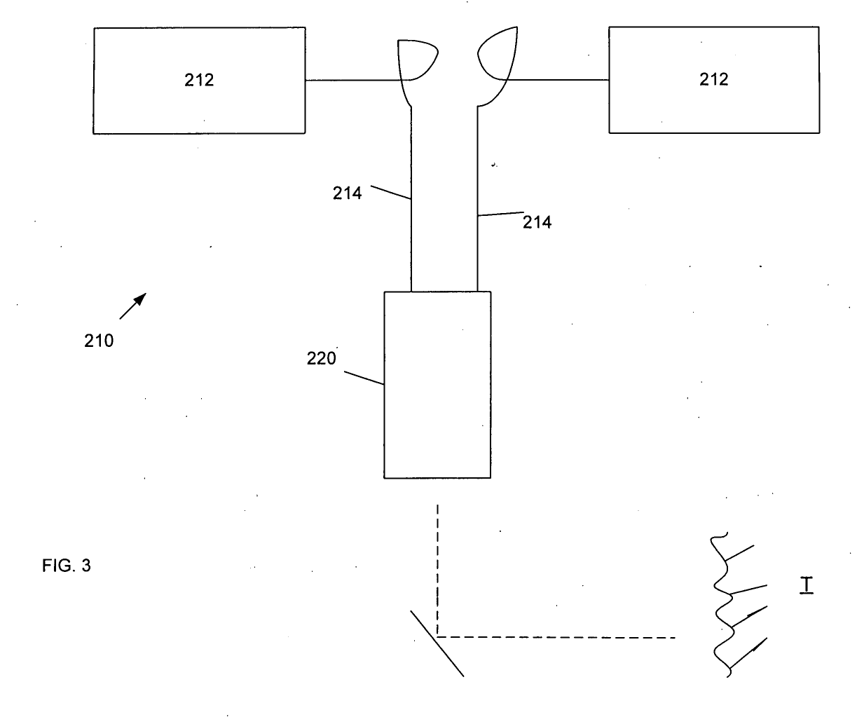

Referring now to FIG. 3, a still further embodiment of the present invention comprises a laser system 210 with multiple fibers 214 of the same or substantially the same core diameters intended for connection to multiple laser sources 212, and coupled to laser delivery device 220, which again can include one or both of a fiber switch, and a spot adjustment device. It should be understood that laser system 210 may be mounted to a handheld device, which can be laser delivery device 220, such as but not limited to an opthalmoscope. In yet another embodiment, laser system 210 includes multiple fibers 214 of different core diameters intended for connection to multiple laser sources 212. It should be understood that additional numbers of lasers sources such as 3, 4, 5, 6, or even more may be coupled to laser system 210. Laser sources 212, as a nonlimiting example, may include yellow, orange, red and infrared laser sources.

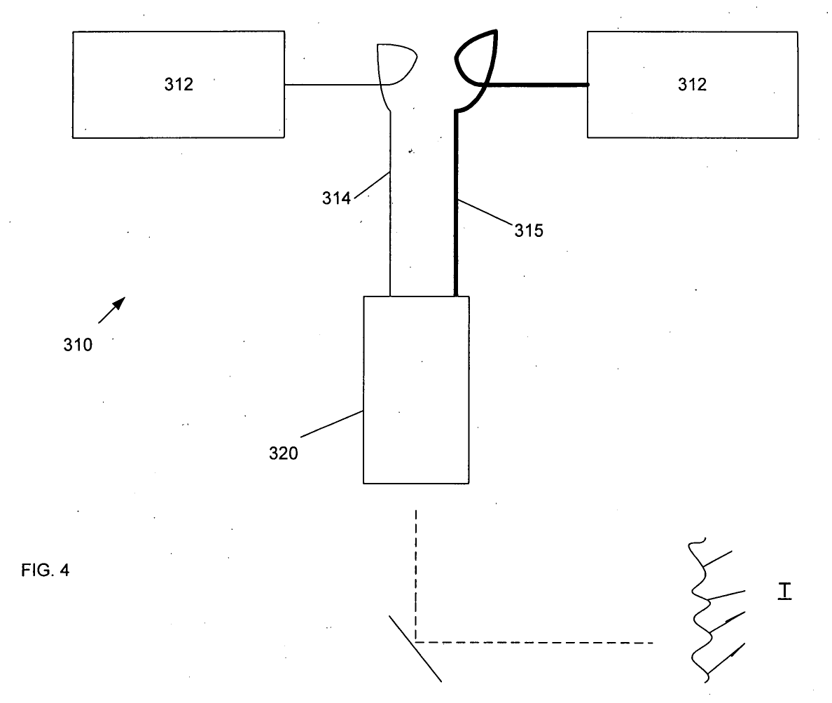

Referring now to FIG. 4, laser delivery system 310, with multiple fibers 314 and 315 of very different diameters, is intended for connection to a single laser source 312 or to multiple laser sources 312, to permit an extremely wide range of treatment parameters. The diameters of fibers 314 and 315 are sufficiently different to create a range of spot sizes greater than 4:1. As a nonlimiting example, they may create a range of spot sizes greater than 10:1, 20:1, 30:1, 40:1, 50:1, 60:1, 70:1, 80:1, 90:1, 100:1 or other ranges. In one embodiment, the spot sizes may range between about 0.060 to 5.00 mm. A laser delivery device 320 is included.

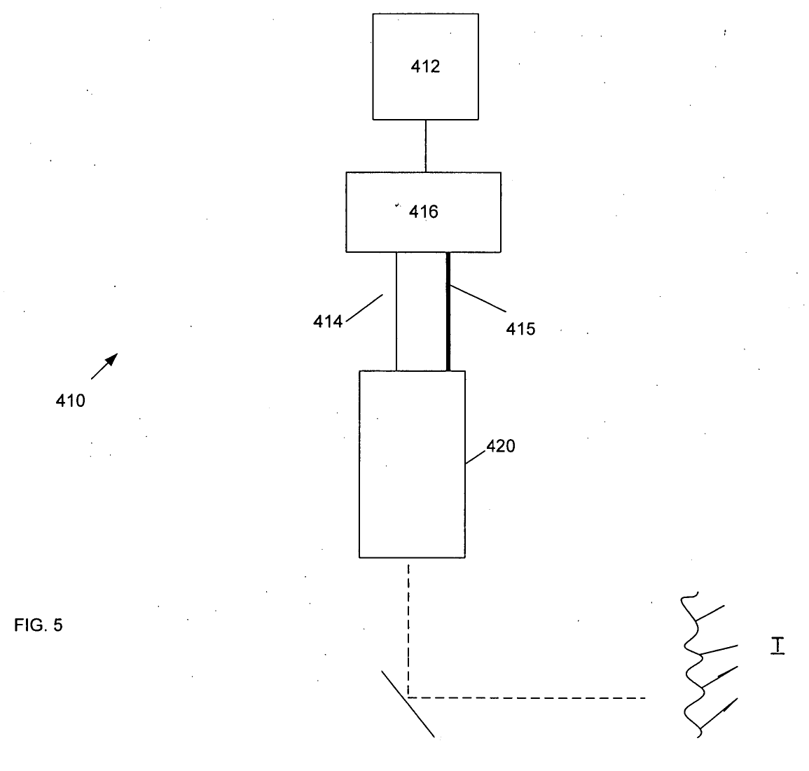

Referring now to FIG. 5, a laser delivery system 410 is provided with multiple fibers 414 and 415 of different core diameters intended for connection to a single laser source 412 capable of emission of multiple laser wavelengths and/or a continuous laser spectrum. A fiber switch 416 may be used to couple fibers 414 and 415 to laser source 412. A laser delivery device 420 is also included.

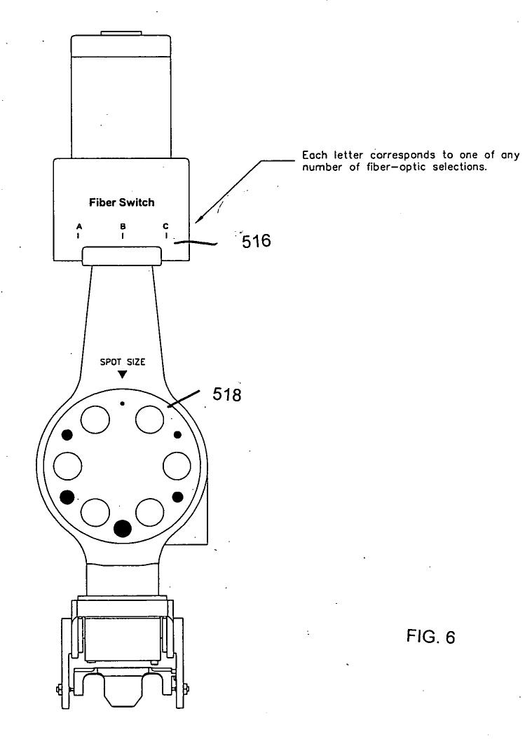

It will be appreciated that in all of the embodiments of the present invention, a fiber switch and a spot size adjustment device can be included. While embodiments of the present invention may associate or incorporate the multiple fibers in the delivery device, it is also possible to associate and incorporate some or all of the fibers in the laser sources. As illustrated in FIG. 6, a spot size adjustment device 518 provides fine control adjustment by the use of discreet spots to image the output of the optical fiber, followed by movement of focusing optics, or finely adjusting to different spot sizes. Fiber switch 516 selects the fiber and provides course adjustment. Course and fine adjustment can be achieved with the same optical elements, without the need for additional optical elements.

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The foregoing description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

1. A laser system, comprising:
at least a first laser source and a second laser source;
at least a first fiber coupled to the first laser source;
at least a second fiber coupled to the second laser source; and
a fiber switching device coupled to the first and second fibers, the fiber switching device configured to provide laser delivery from each of the first and second fibers without additional optical alignment.

2. The system of claim 1, further comprising:
a laser delivery device coupled to at least one of the first and second fibers.

3. The system of claim 2, wherein the laser delivery device is selected from, a laser slit lamp adapter, an indirect ophthalmoscope, a laser operating microscope, a direct ophthalmoscope, an intraocular probe, a scanning laser ophthalmoscope, a fundus camera, a laparascope, an endoscope, a microscope, and a handheld laser delivery device.

4. The system of claim 1, further comprising:
a spot size adjustment device coupled to at least one of the first and second fibers.

5. The system of claim 4, wherein the spot size adjustment device provides automatic sensing of at least one of an output end or an input end of the first and second fibers.

6. The system of claim 2, wherein the laser delivery device includes a spot size adjustment device

7. The system of claim 1, wherein the first and second fibers have different diameters.

8. The system of claim 1, wherein the fiber switching mechanism is selected from, manual, electromechanical and optomechanical.

9. The system of claim 1, wherein the first and a second laser sources are selected from a, diode laser, ion laser, dye laser, Ti:sapphire laser, Alexandrite laser, and solid state laser.

10. A laser system comprising:
at least a first laser source and a second laser source;
at least a first fiber coupled to the first laser source;
at least a second fiber coupled to the second laser source; and
a fiber switching device coupled to the first and second fibers, the fiber switching device configured to provide repositioning of and laser delivery from each of the first and second fibers without additional optical alignment.

11. The system of claim 10, further comprising:
a laser delivery device coupled to at least one of the first and second fibers.

12. The system of claim 11, wherein the laser delivery device is selected from, a laser slit lamp adapter, an indirect ophthalmoscope, a laser operating microscope, a direct ophthalmoscope, an intraocular probe, a scanning laser ophthalmoscope, a fundus camera, a laparascope, an endoscope, a microscope, and a handheld laser delivery device.

13. The system of claim 10, further comprising:
a spot size adjustment device coupled to at least one of the first and second fibers.

14. The system of claim 13, wherein the spot size adjustment device provides automatic sensing of at least one of an output end or an input end of the first and second fibers.

15. The system of claim 11, wherein the laser delivery device includes a spot size adjustment device

16. A laser system comprising:
a laser source;
at least a first fiber capable of being coupled to the laser source;
at least a second fiber capable of being coupled to the laser source; and
a fiber switching mechanism configured to provide laser delivery from each of the first and second fibers without the need for additional optical alignment,

17. The system of claim 16, further comprising:
a laser delivery device coupled to at least one of the first and second fibers.

18. The system of claim 17, wherein the laser delivery device is selected from, a laser slit lamp adapter, an indirect ophthalmoscope, a laser operating microscope, a direct ophthalmoscope, an intraocular probe, a scanning laser ophthalmoscope, a fundus camera, a laparascope, an endoscope, a microscope, and a handheld laser delivery device.

19. The system of claim 16, further comprising:
a spot size adjustment device coupled to at least one of the first and second fibers.

20. The system of claim 19, wherein the spot size adjustment device provides automatic sensing of at least one of an output end or an input end of the first and second fibers.

21. The system of claim 17, wherein the laser delivery device includes a spot size adjustment device.

22. The system of claim 16, wherein the first and second fibers have different diameters.

23. The system of claim 16, wherein the fiber switching mechanism is selected from, manual, electromechanical and optomechanical.

24. The system of claim 16, wherein the first and a second laser sources are selected from a, diode laser, ion laser, dye laser, Ti:sapphire laser, Alexandrite laser, and solid state laser.

25. A laser system comprising:
a laser source;
at least a first fiber capable of being coupled to the laser source;
at least a second fiber capable of being coupled to the laser source;
a fiber switching mechanism configured to provide repositioning of and laser delivery from each of the first and second fibers without the need for additional optical alignment, and
a spot size adjustment device coupled to at least one of the first and second fibers.

26. The system of claim 25, further comprising:
a laser delivery device coupled to at least one of the first and second fibers.

27. The system of claim 26, wherein the laser delivery device is selected from, a laser slit lamp adapter, an indirect ophthalmoscope, a laser operating microscope, a direct ophthalmoscope, an intraocular probe, a scanning laser ophthalmoscope, a fundus camera, a laparascope, an endoscope, a microscope, and a handheld laser delivery device.

28. The system of claim 26, wherein the further comprising:
a spot size adjustment device is coupled to the laser delivery device.

29. The system of claim 25, wherein the spot size adjustment device provides automatic sensing of at least one of an output end or an input end of the first and second fibers.

30. The system of claim 25, wherein the first and second fibers have different diameters.

31. The system of claim 25, wherein the fiber switching mechanism is selected from, manual, electromechanical and optomechanical.

32. The system of claim 25, wherein the first and a second laser sources are selected from a, diode laser, ion laser, dye laser, Ti:sapphire laser, Alexandrite laser, and solid state laser.

33. A laser system, comprising:
a slit lamp adapter;
a plurality of laser sources;
a plurality of fibers, wherein at least one of the fibers is coupled to one of the laser sources and another of the fibers is coupled to another of the laser sources, wherein light from said laser sources are coupled to the slit lamp adapter;
wherein the plurality of fibers have core diameters that are the same.

34. The system of claim 33 further comprising a spot size adjustment device is coupled to the fibers.

35. The system of claim 33 further comprising a fiber switching mechanism configured to provide repositioning of and laser delivery from each of the fibers without the need for additional optical alignment.

36. The system of claim 33 further comprising a fiber sensing mechanism.

37. A laser system, comprising:
a plurality of laser sources;
a plurality of fibers, wherein at least one of the fibers is coupled to one of the laser sources and another of the fibers is coupled to another of the laser sources;
wherein the plurality of fibers each have different core diameters.

38. The system of claim 37 further comprising:
a spot size adjustment device coupled to the fibers.

39. The system of claim 37 further comprising a fiber switching mechanism configured to provide repositioning of and laser delivery from each of the fibers without the need for additional optical alignment.

40. The system of claim 37 further comprising a fiber sensing mechanism.

41. A laser system, comprising:
a single laser source;
a plurality of fibers, wherein at least one of the fibers is coupled to the laser source and at least another of the fibers is also coupled to the laser source;
wherein the plurality of fibers have each have a different core diameter, said fibers each create a different spot size from the laser source and said fibers create a range of spot sizes greater than 20:1.

42. The system of claim 41, further comprising a spot size adjustment device is coupled to the fibers.

43. The system of claim 41, further comprising a fiber switching mechanism configured to provide repositioning of and laser delivery from each of the fibers without the need for additional optical alignment.

44. The system of claim 41, further comprising a fiber sensing mechanism.

45. A laser system, comprising:
a single laser source capable of emission of multiple laser wavelengths;
a plurality of fibers each coupled to the single laser source, light from the single laser source being coupled to a laser delivery device; and
wherein the plurality of fibers have each have a different core diameter.

46. The system of claim 45 further comprising, a spot size adjustment device coupled to the fibers.

47. The system of claim 45 further comprising, a fiber switching mechanism configured to provide repositioning of the fibers without the need for additional optical alignment.

48. The system of claim 45, further comprising, a moveable input lens that couples a selected fiber for delivery of a beam from the single laser source.

49. The system of claim 45 further comprising a fiber sensing mechanism.

50. The system of claim 45 wherein the laser source provides a continuous laser spectrum,

51. A laser system, comprising:
a laser source;
a fiber switch coupled to the laser source;
at least first and second fibers coupled to the fiber switch; the first and second fibers having very different core diameters; and
a laser delivery device coupled to the first and second fibers.
